(19) United States
(12) Patent Application Publication
(10) Pub. No.: US 2025/0270526 A1
(43) Pub. Date: Aug. 28, 2025

(54) ARTIFICIALLY ENHANCED BIODEGRADATION OF POLYETHYLENE TEREPHTHALATE

(71) Applicant: Elizabeth Hanechak, Russell, MA (US)

(72) Inventor: Elizabeth Hanechak, Russell, MA (US)

(21) Appl. No.: 18/584,173

(22) Filed: Feb. 22, 2024

(51) Int. Cl. C12N 9/18; C08J 11/10

(52) U.S. Cl. CPC C12N 9/18; C08J 11/105; C12Y 301/01074

(57) ABSTRACT

Presented herein is an artificial cutinase polypeptide in which the first C31-C109 disulfide bridge is deleted, resulting in an increase in enzymatic activity. In certain embodiments C31 is mutated to an Alanine or a Serine. Further presented are means of creating and using said cutinase for the purposes of degrading PET.

### SUBMISSION OF SEQUENCE LISTING

The instant application contains a Sequence Listing which has been submitted electronically in XML format and is hereby incorporated by reference in its entirety. Said.xml file, created on 20 Feb. 2024, is named “EZH0001US-SEQs.xml” and is approximately 6,617 bytes in size. The content of the XML file, compliant with the ST.26 format and incorporated herein by reference, is a computer readable form (CRF) of ten (10) sequences:

SEQ ID NO:1 is an example nucleic acid sequence of a wildtype cutinase.
SEQ ID NO:2 is a forward nucleic acid primer for the C31A mutation.
SEQ ID NO:3 is a reverse nucleic acid primer for the C31A mutation.
SEQ ID NO:4 is a forward nucleic acid primer for the C31S mutation.
SEQ ID NO:5 is a reverse nucleic acid primer for the C31S mutation.
SEQ ID NO:6 is an example synthetic nucleic acid sequence of the C31A mutation.
SEQ ID NO:7 is an example synthetic nucleic acid sequence of the C31S mutation.
SEQ ID NO:8 is an example amino acid sequence for the nucleic acid sequence of SEQ ID NO:1.
SEQ ID NO:9 is an example amino acid sequence for the nucleic acid sequence of SEQ ID NO:6.
SEQ ID NO:10 is an example amino acid sequence for the nucleic acid sequence of SEQ ID NO:7.
The Sequence Listing does not go beyond the disclosure in the application as filed.

### BACKGROUND OF THE INVENTION

### Field of the Invention

This disclosure provides a means of enhanced biodegradation of polyethylene terephthalate. Provided is an artificial cutinase enzyme created through site-directed mutagenesis adapted for the biodegradation of polyethylene terephthalate (PET) and methods and means of usage thereof.

### BRIEF DESCRIPTION OF THE ART

Plastic pollution is one of the largest issues facing society today. Plastic is environmentally destructive from the beginning of its lifespan. For example, plastic production releases over 850 million metric tons of greenhouse gas emissions every year. The artificial chemical composition of plastics results in materials that do not readily degrade in the environment. Since 1950, plastics production generated over 8.3 billion metric tons of plastic waste. Severe environmental consequences result from plastic accumulation. In 2010, 8 million tons of plastic waste was deposited into the ocean, and it is estimated that plastic kills over 1 million marine animals each year. The plastics pollution is further compounded by mechanical breakdown of larger plastic pieces into microplastic particles found throughout the environment. Plastic pollution also has severe economic effects. In 2019, the United States spent over $32 billion on collecting, sorting, and recycling plastic waste; this figure does not account for environmental cleanup costs or follow-on costs to human health.

Polyethylene terephthalate (PET), one of the most common types of plastic used for single-use water bottles and food packaging, is produced through the polymerization of the organic compounds ethylene glycol and terephthalic acid. A hydroxyl group on the ethylene glycol reacts with a carboxyl group on the terephthalic acid at a high temperature, forming ester groups that link PET units into polymers. PET has the IUPAC name of poly(ethylene terephthalate) and is sold under the trade names of Terylene® and Dacron®. Although ester bonds are found in nature, the artificial PET-based polymer bonds are difficult to break, and thus the polymers are difficult to naturally degrade, as there are very few mechanisms (enzymes, proteins, catalysts, etc.) found in nature that can break the bonds, let alone evolved to do so in optimal fashion.

There are multiple proposed solutions to the plastic problem, including recycling, incineration, and biodegradation. Recycling is not viable. According to a 2022 review of the plastic crisis completed by Greenpeace, an international organization fighting against climate change and pollution, “Mechanical and chemical recycling of plastic waste has largely failed and will always fail because plastic waste is: (1) extremely difficult to collect, (2) virtually impossible to sort for recycling, (3) environmentally harmful to reprocess, (4) often made of and contaminated by toxic materials, and (5) not economical to recycle.” Less than 9% of recyclable plastics were actually recycled in 2018, and less than 4% of the 51 million tons of household plastic waste was recycled in 2021.

Incineration is also not viable. Burning plastic releases toxic chemicals, including air pollutants, heavy metals, and carcinogens. The adverse health effects of incineration byproducts on humans are well documented by multiple studies. It was found that residents of a town in Korea, living in close proximity to three plastic waste incinerators, were at higher risk for developing cancer: In another study, the risks for gallbladder cancer among males and kidney cancer among females were 2.65 and 2.82 times higher in the exposed group versus the respective control group. In women living close to incinerators in Cumbria, England, studies documented an increased risk of lethal congenital anomalies; in particular, spina bifida and heart defects.

Biodegradation, the use of naturally occurring microorganisms to break down materials, is a promising approach to the plastic problem. Some consider biodegradation to be the most accepted, eco-friendly, and cost-effective method of polyethylene waste disposal. Most natural organisms lack the cellular machinery needed to break down the man-made chemical bonds forming plastics. In 2016, a group of Japanese scientists discovered a novel bacteria species, Ideonella sakaiensis, outside of a plastic waste facility. I. sakaiensis hydrolyzed polyethylene terephthalate (PET) samples in a matter of days, using the plastic as its main carbon source. In another study, eight fungal species native to Indian mangrove soil, including Aspergillus glaucus, degraded 28.80% of polyethylene and 8.16% of other plastics in a one-month period. Algal species are also studied as potential plastic eaters, specifically for use in marine environments polluted by microplastics. One study found that algae, particularly microalgae, can degrade the plastic materials through the toxins or enzymes synthesized by microalgae which then utilize the plastic polymers as carbon sources.

Microbes that can biodegrade plastics typically secrete enzymes to degrade PET. Enzymes are specialized protein structures that catalyze chemical reactions. I. sakaiensis secretes two enzymes, PETase and MHETase, to biodegrade plastic. One fungus, Fusarium solani pisi, secretes an enzyme demonstrated capable of degrading plastic.

Fusarium solani pisi secretes a cutinase, a serine esterase that degrades the naturally occurring polyester cutin, which is found in plant cell walls. Cutin and PET share a similar structural makeup; namely, in that the monomers of cutin are linked by ester bonds. In a review which analyzed and assayed multiple PET hydrolytic enzymes, cutinase was found to be the most effective in degrading a PET sample. Despite these encouraging properties, cutinase has low catalytic power and turnover rates which prohibit cutinase development for large-scale application as a plastic pollution waste-management system.

Thus, there is a need for an improved cutinase enzyme with enhanced catalytic power.

### BRIEF SUMMARY OF THE INVENTION

This disclosure provides for an artificial cutinase polypeptide. In embodiments, the artificial cutinase polypeptide has the C31-C109 disulfide bridge, hereinafter referred to as a “first disulfide bridge,” deleted. In certain embodiments, the cutinase polypeptide is lyophilized. In still other embodiments the cutinase polypeptide is in a solution with at least one stabilizer.

In other embodiments of the disclosure, there is an artificial plasmid that encodes a cutinase polypeptide sequence wherein C31 is mutated in comparison to the wildtype sequence. In certain embodiments, the C31 is mutated to an Alanine. In still other embodiments, the C31 is mutated to a Serine.

In certain embodiments an organism is artificially cultivated and transfected with the artificial cutinase plasmid. In some embodiments, the organism is grown in a bioreactor. In still other embodiments, that bioreactor may be an open-air bioreactor.

In additional embodiments, a method of PET degradation is conveyed the method comprising, exposing a PET article to an artificial cutinase enzyme as described herein. In continued embodiments of the method, the artificial cutinase polypeptide is contained and/or secreted by the artificially cultivated transfected organism. In still other embodiments of the method, the exposure of the PET article is accomplished via a solution containing the artificial cutinase enzyme. In still other embodiments of the method, the solution further comprises at least one selected from the group of: a surfactant, a pH buffer, and an enzyme stabilizer. In another embodiment of the method, the resulting waste stream after degradation is subjected to further treatment.

In another embodiment, there is the artificial cutinase polynucleotide of SEQ ID NO: 1 encoding a polypeptide having at least 40% sequence identity with a conserved active site, wherein C31 is mutated to an Alanine or Serine.

In another embodiment, there is the artificial cutinase polypeptide having the polypeptide sequence of SEQ ID NO:9, as well as a nucleic acid sequence that encodes the polypeptide sequence of SEQ ID NO:9.

In another embodiment, there is the artificial cutinase polypeptide having the polypeptide sequence of SEQ ID NO:10, as well as a nucleic acid sequence that encodes the polypeptide sequence of SEQ ID NO:10.

### BRIEF DESCRIPTION OF THE DRAWINGS

FIG. 1A illustrates the wildtype cutinase structure with residues C31 and C109 highlighted. These two residues form a disulfide bridge, which contributes to the cutinase thermal stability. FIG. 1B displays the mutant cutinase which changes C31 to alanine and C109 to serine. FIG. 1C shows the mutant cutinase which changes C31 to serine and C109 to serine.

### DETAILED DESCRIPTION OF THE INVENTION

### Terminology

Unless defined otherwise, all technical and scientific terms used herein have the same meaning as is commonly understood by one of skill in the art to which the claimed subject matter and this disclosure belongs. Singleton et al., Dictionary of Microbiology and Molecular Biology, 2nd Ed., John Wiley and Sons, New York (1994), and Hale and Marham, The Harper Collins Dictionary of Biology, Harper Perennial, New York (1991), provide dictionary definitions of many of the terms used in the disclosure. It is to be understood that the foregoing general description and the following detailed description are exemplary and explanatory only and are not restrictive of any subject matter claimed.

In this disclosure, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The transitional phrases “comprising” and “having” are open-ended and may include other, not explicitly recited, elements. An element or a plurality of elements having a particular property may include additional elements not having that property.

Embodiments disclosed with an open-ended transitional phrase such as “comprising” include, as alternative embodiments, embodiments recited with the same elements but with an intermediate or closed transitional phrase such as “consisting essentially of” or “consisting of.” As used herein, ranges and amounts can be expressed as “about” a particular value or range. About also includes the exact amount. Hence “about 5 μL” means “about 5 μL” and “5 μL.” Generally, the term “about” includes an amount that would be expected to be within experimental error or within the error expected from manufacturing, production, or experimental tolerances.

The terms “or/and” and “and/or” mean “either . . . or . . . , or both . . . and . . . ” when referring to two elements, and mean “either . . . , . . . or . . . , or any combination or all thereof” when referring to three or more elements. As an example, the phrase “A or/and B” means “either A or B, or both A and B”, and the phrase “A, B or/and C” means “either A, B or C, or any combination or all thereof”.

As used in the specification and the claims, all transitional terms such as “comprising”, “containing”, “having”, “including”, “possessing”, “holding”, “carrying”, “bearing”, “composed of”, “characterized by” and the like are open-ended and inclusive, that is, mean including but not limited to and do not exclude additional, unrecited element(s) or method step(s). Only the transitional term “consisting of” is closed, that is, excludes any additional, unrecited element or method step, and the transitional term “consisting essentially of” is semi-closed, that is, only allows inclusion of additional, unrecited element(s) or method step(s) that do not materially affect the basic and novel characteristic(s) of that particular embodiment.

The term “exemplary” as used herein means “serving as an example, instance or illustration”. Any embodiment or feature characterized herein as “exemplary” is not necessarily to be construed as preferred or advantageous over other embodiments or features.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

Unless otherwise indicated, nucleic acid sequences are written left to right in the 5′ to 3′ direction, and amino acid sequences are written left to right in the amino (N-terminus) to carboxy (C-terminus) direction. The terms defined below are more fully defined by reference to the specification as a whole.

As used herein in the specification and in the claims, the terms “polynucleotide”, “oligonucleotide”, “nucleic acid” and “nucleic acid molecule” are used interchangeably herein to refer to a polymeric form of nucleotides of any length, and may comprise ribonucleotides, deoxyribonucleotides, analogs thereof, or mixtures thereof. These terms refer only to the primary structure of the molecule. Thus, the terms include triple-, double- and single-stranded deoxyribonucleic acid (“DNA”), as well as triple-, double- and single-stranded ribonucleic acid (“RNA”). They also include modified, for example by alkylation and/or by capping, and unmodified forms of the polynucleotide. More particularly, the terms “polynucleotide”, “oligonucleotide”, “nucleic acid” and “nucleic acid molecule” include polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing nonnucleotidic backbones, for example, polyamide (e.g., peptide nucleic acids (“PNAs”) and polymorpholino (commercially available from the Anti-Virals, Inc., Corvallis, Oregon, as Neugene), and other synthetic sequence-specific nucleic acid polymers provided that the polymers contain nucleobases in a configuration which allows for base pairing and base stacking, such as is found in DNA and RNA. Thus, these terms include, for example, 3′-deoxy-2′, 5′-DNA, oligodeoxyribonucleotide N3′ P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps”, substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g., nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

As used herein in the specification and in the claims, the terms “polynucleotide”, “oligonucleotide”, “nucleic acid”, “nucleic acid molecule” and “gene” can refer to the entire sequence or gene or a fragment thereof, such as a functional fragment. The terms “polypeptide”, “peptide” and “protein” can refer to the entire amino acid sequence or a fragment thereof, such as a functional fragment. The term “enzyme” generally refers to a polypeptide folded into a three-dimensional structure.

As used herein, and unless the context suggests otherwise, the term “bind” or “binds” refers to the ability of molecular entities to interact via, e.g., hydrophobic bonds, Van der Waals forces, ionic interactions or a combination thereof, wherein the interaction is sufficiently stable (i.e., the dissociation constant or Kd is low enough) under biologically relevant conditions (e.g., temperature, ionic, and pH) to effectuate the desired function or activity. For example, in certain embodiments the Kd will be less than about 1000, 100, 10, 0.1, 0.01, 0.001, 0.0001, 0.00001 or 0.0000001 μM. The above is distinguished from, e.g., a covalent bond, in which two atoms share one or more electron pairs.

As used herein in the specification and in the claims, the phrase “nucleic acid sequence” refers to a contiguous nucleic acid sequence. The sequence can be either single-stranded or double-stranded, and can contain DNA residues, RNA residues, or nucleic acid analogs, or any combination thereof. The sequence can be a polynucleotide of, e.g., from about 2 bp to about 50 kbp in length, e.g., about 2 bp to 5 kb, 2 bp to 4 kb, 2 bp to 3 kb, 2 bp to 2 kb, 2 bp to 1 kb, 2 bp to 500 bp, 2 bp to 100 bp, 2 bp to 50 bp, and values in between, and the corresponding ranges of nucleotides in length and values in between, to a full-length genomic sequence of thousands or hundreds of thousands of base pairs.

The term “coding sequence” or “coding region” means a polynucleotide which directly specifies the amino acid sequence of a polypeptide. The boundaries of the coding sequence are generally determined by an open reading frame which begins with a start codon such as ATG, GTG, or TTG and ends with a stop codon such as TAA, TAG, or TGA. The coding sequence may be a genomic DNA, cDNA, synthetic DNA, or a combination thereof.

Where the polynucleotides are to be used to express encoded proteins, nucleotides that can perform that function or which can be modified (e.g., reverse transcribed) to perform that function are used. Where the polynucleotides are to be used in a scheme that requires that a complementary strand be formed to a given polynucleotide, nucleotides are used which permit such formation.

The term “control sequences” means nucleic acid sequences necessary for expression of a polynucleotide encoding a mature polypeptide of the present invention. Each control sequence may be native (i.e. from the same gene) or foreign (i.e. from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

The term “subsequence” means a polynucleotide having one or more (e.g. several) nucleotides absent from the 5′ and/or 3′ end of a mature polypeptide coding sequence; wherein the subsequence encodes a fragment having enzymatic activity, such as activity on PET.

The term “corresponding to” as used herein, refers to a way of determining the specific amino acid of a sequence wherein reference is made to a specific amino acid sequence. E.g. for the purposes of the present disclosure, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NO:2, or any other amino acid sequence listed herein, has been described elsewhere herein. Alternative alignment methods, such as computer-based BLAST alignment algorithms, may be used, and are well-known for the skilled person.

The term “expression” includes any step involved in the production of a polypeptide including, but not limited to, transcription, post-transcriptional modification, translation, post-translational modification, and secretion.

The term “expression vector” means a linear or circular DNA molecule that comprises a polynucleotide encoding a polypeptide and is operably linked to control sequences that provide for its expression.

The term “fragment” means a polypeptide having one or more (e.g. several) amino acids absent from the amino and/or carboxyl terminus of a mature polypeptide; wherein the fragment has endoglucanase activity. In one aspect, a fragment contains at least 85%, 86%, 87%, 88%, 89%, 90%, 91%, 92%, 93%, 94% or 95% of the number of amino acids of the mature polypeptide.

The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

The term “improved property” means a characteristic associated with a variant that is improved compared to the parent. Such improved properties include, but are not limited to, catalytic efficiency, catalytic rate, chemical stability, oxidation stability, pH activity, pH stability, specific activity, stability under storage conditions, chelator stability, substrate binding, substrate cleavage, substrate specificity, substrate stability, surface properties, thermal activity, and thermostability.

The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g. multiple copies of a gene encoding the substance; use of a stronger promoter than the promoter naturally associated with the gene encoding the substance). An isolated substance may be present in a fermentation broth sample.

The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e. with a different C-terminal and/or N-terminal amino acid) expressed by the same polynucleotide. It is also known in the art that different host cells process polypeptides differently, and thus, one host cell expressing a polynucleotide may produce a different mature polypeptide (e.g. having a different C-terminal and/or N-terminal amino acid) as compared to another host cell expressing the same polynucleotide.

The term “mature polypeptide coding sequence” means a polynucleotide that encodes a mature polypeptide having enzymatic activity such as activity on xanthan gum pretreated with xanthan lyase or xanthan lyase activity.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., where one or more of the hydroxyl groups are replaced with halogen, aliphatic groups, or is functionalized as ethers, amines, or the like. The term “nucleotidic unit” is intended to encompass nucleosides and nucleotides.

Standard A-T and G-C base pairs form under conditions which allow the formation of hydrogen bonds between the N3-H and C4-oxy of thymidine and the NI and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) may be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al., hereby incorporated by reference in its entirety). Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al., 1993, J. Am. Chem. Soc. 115:4461-4467 and references cited therein; and isoguanine nucleotides may be prepared using the method described by Switzer et al., 1993, supra, and Mantsch et al., 1993, Biochem. 14:5593-5601, or by the method described in U.S. Pat. No. 5,780,610 to Collins et al., each of which is hereby incorporated by reference in its entirety. Other nonnatural base pairs may be synthesized by the method described in Piccirilli et al., 1990, Nature 343:33-37, hereby incorporated by reference in its entirety, which describes the synthesis of 2,6-diaminopyrimidine and its complement (1-methylpyrazolo-[4,3]pyrimidine-5,7-(4H,6H)-dione. Other such modified nucleotidic units which form unique base pairs are known, such as those described in Leach et al. (1992) J. Am. Chem. Soc. 114:3675-3683 and Switzer et al., supra.

The term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Complementarity of polynucleotides typically refers to C/G and A/T (or U) base pairings between antiparallel DNA/DNA, DNA/RNA or RNA/RNA sequences. Thus, the polynucleotide whose sequence is 5′-TATAC-3′ is complementary to a polynucleotide whose sequence is 5′-GTATA-3′. A nucleotide sequence is “substantially complementary” to a reference nucleotide sequence if the sequence complementary to the subject nucleotide sequence is substantially identical to the reference nucleotide sequence.

The term “hybridizing specifically to”, “specific hybridization” or “selectively hybridize to” refers to the binding, duplexing or hybridizing of a polynucleotide preferentially to a particular nucleotide sequence under stringent conditions (e.g., highly stringent conditions) when that sequence is present in a mixture of (e.g., total cellular) DNA or/and RNA.

As used herein in the specification and in the claims, the term “vector” or “DNA vector” refers to a DNA sequence that is used to perform a “carrying” function for another polynucletiode. For example, vectors are often used to allow a polynucleotide to be propagated within a living cell.

A “host”, as the term is used herein, includes prokaryotic or eukaryotic organisms that can be genetically engineered. For examples of such hosts, see Maniatis et al., Molecular Cloning. A Laboratory Manual, Cold Spring Harbor Laboratory, Cold Spring Harbor, N.Y. (1982). As used herein, the terms “host”, “host cell”, “host system” and “expression host” are used interchangeably.

As used herein in the specification and in the claims, the phrase “predetermined time period” refers to a specified amount of time. A “predetermined period of time” can be on the order of seconds, minutes, hours, days, weeks, or months. For example, a “predetermined time period” can be between 1 and 59 minutes, or any increment between 1 and 2 hours, or any increment between 2 and 4 hours, or any increment between 4 and 6 hours, or any increment between 6 and 12 hours, or any increment between 12 and 24 hours, or any increment between 1 day and 2 days, or any increment between 2 days and 4 days, and any increment between 4 days and 7 days, and any increment between 1 week and 4 weeks, and any increment between 1 month and 12 months, or any combination of incremental time periods therein.

As used herein, the term “expression marker” are used to determine whether the gene or nucleic acid of interest is expressed in a cell or has been activated in a cell. Expression markers typically have easily identifiable characteristics, such as fluorescence, or easily assayed products, such as an enzyme. Expression markers can also confer antibiotic resistance to a host cell or tissue. Expression markers include, for example, labels such as green fluorescent protein (GFP or eGFP) or other fluorescence genes, luciferase, β-galactosidase and alkaline phosphatase.

As used herein, the term “promoter” is an array of nucleic acid control sequences which direct transcription of a nucleic acid. A promoter includes necessary nucleic acid sequences near the start site of transcription. A promoter also optionally includes distal enhancer or repressor elements which can be located as much as several thousand base pairs from the start site of transcription. In any aspect or embodiment described herein, the promoter is a constitutively active promoter. In any aspect or embodiment described herein, the promoter is an inducible promoter. In any aspect or embodiment described herein, the protein and/or the coded polynucleotide is under expression or transcription control of (e.g., operatively connected or linked to) a human promoter or a promoter of a different species (e.g., a viral promoter, a prokaryotic promoter, or a eukaryotic promoter, for example, a promoter from a non-human primate, a rodent, a dog, a cat, a pig, a cow, a horse, or other species). In any aspect or embodiment described herein, the promoter may be a tissue or cell specific promoter.

In some embodiments, the terms “substantially pure” and “isolated” mean that an object macromolecular species is the predominant macromolecular species present on a molar or weight basis (i.e., on a molar or weight basis, it is more abundant than any other individual macromolecular species in the composition). In some embodiments, a substantially pure composition is one in which an object macromolecular species constitutes at least about 50% on a molar or weight basis of all macromolecular species present. In some embodiments, a substantially pure or isolated macromolecular species constitutes at least about 50%, 60%, 65%, 70%, 75%, 80%, 85%, 90%, 95% or 98% of the macromolecular species present on a molar or weight basis. In certain embodiments, a substantially pure composition means that at least about 70%, 75%, 80%, 85%, 90%, 95% or 98% of the macromolecular species present in the composition, on a molar or weight basis, is the macromolecular species of interest. In further embodiments, an object macromolecular species is purified to essential homogeneity (i.e., contaminant macromolecular species cannot be detected in the composition by conventional detection methods) if the composition consists essentially of a single macromolecular species (e.g., at least about 95%, 96%, 97%, 98% or 99% of the object macromolecular species on a molar or weight basis). Solvent molecules, elemental ion species, small molecules (≤about 500 Daltons), and stabilizers (e.g., bovine serum albumin [BSA]) are not considered (contaminant) macromolecular species for purposes of these embodiments.

In describing sequence comparisons, several different terms are commonly used: “identity,” “similarity,” and “homology.” Even though they are often used interchangeably, they have quite different meanings. Sequence identity refers to the occurrence of exactly the same nucleotide or amino acid in the same position in aligned sequences. Sequence similarity takes approximate matches into account and is meaningful only when such substitutions are scored according to some measure of ‘difference’ or ‘sameness’ with conservative or highly probably substitutions assigned more favorable scores than non-conservative or unlikely ones. The term ‘sequence homology’ is often the most misapplied term. By definition, when it is asserted that sequence A has high homology to sequence B, two distinct claims are made: not only that sequences A and B look much the same, but also that all of their ancestors also looked the same, going all the way back to a common ancestor. Although the first of these claims is easily verified, the second is frequently in doubt. Although the comparison of two sequences is often summarized as a percentage sequence homology, that usage is generally incorrect as the value really indicates identity and/or similarity and does not necessarily reflect an evolutionary relationship. Nevertheless, one of ordinary skill in the art can, and often does, use the terms interchangeably and may do so herein unless specified otherwise or the context otherwise indicates that a more specific reading of the term is required. Further, in the terms “sequence identity” or “percent sequence identity,” refer to the degree with which two peptide chain listings may be found to be similar or differ. It is commonly understood that sequences with a greater sequence identity or higher percent identity are likely to have similar, if not identical, function. Indeed, conserved sequence elements, such as those that define a structural motif or an active site are even more likely to maintain functionality of an enzyme despite lower overall percentages of sequence identity. Performance of the same function, however, does not necessarily guarantee the same rate of reaction, the same thermal stability, or the ability to handle a particular substrate. It is known that homologous sequences that share more than 40% identity are very likely to share functional similarity, particularly if the active site sequences are conserved. In embodiments of the present disclosure, enzymes with sequences of 40% or greater that preserve the active site are expressly contemplated. In particular, sequence identities of 40-50%, 51-60%, 61-70%, 71-80%, and 81-100% are all possible.

For purposes of the present disclosure, the nucleotide sequence disclosed in SEQ ID NO: 1 may be used to determine the corresponding nucleotide sequence or amino acid residue in another cutinase and the sequences disclosed in SEQ IDs NOs: 2-5 may be used to determine the corresponding nucleotide sequence or amino acid residue in another cutinase. The amino acid or nucleic acid sequence of another cutinase is aligned with the sequence disclosed in SEQ ID NO: 1 or SEQ IDs NOs: 2-5, and based on the alignment, the amino acid position number corresponding to any amino acid residue in the mature polypeptide disclosed in SEQ ID NO: 1-5 is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et at., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another cutinase can be determined by an alignment of multiple polypeptide sequences using several computer programs including, but not limited to, MUSCLE (multiple sequence comparison by log-expectation; version 3.5 or later; Edgar, 2004, Nucleic Acids Research 32:1792-1797), MAFFT (version 6.857 or later; Katoh and Kuma, 2002, Nucleic Acids Research 30:3059-3066; Katoh et al., 2005, Nucleic Acids Research 33:511-518; Katoh and Toh, 2007, Bioinformatics 23:372-374; Katoh et al., 2009, Methods in Molecular Biology 537:39-64, Katoh and Toh, 2010, Bioinformatics 26:1899-1900), and EMBOSS EMMA employing ClustalW (1.83 or later; Thompson et al., 1994, Nucleic Acids Research 22:4673-4680), using their respective default parameters.

When the other enzyme has diverged from the mature polypeptide encoded by the nucleic acid sequence of SEQ ID NO:1 such that traditional sequence-based comparison fails to detect their relationship (Lindahl and Elofsson, 2000, J. Mol. Biol. 295:613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Altschul et al., 1997, Nucleic Acids Res. 25:3389-3402). Even greater sensitivity can be achieved if the family or superfamily for the polypeptide has one or more representatives in the protein structure databases. Programs such as GenTHREADER (Jones, 1999, J. Mol. Biol. 287:797-815; McGuffin and Jones, 2003, Bioinformatics 19:874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313:903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33:88-96) or combinatorial extension (Shindyalov and Bourne, 1998, Protein Engineering 1 (1): 739-747), and implementation of these algorithms can additionally be utilized to query structure databases with a structure of interest in order to discover possible structural homologs (e.g. Holm and Park, 2000, Bioinformatics 16:566-567).

If not explicitly indicated otherwise, all the variants described herein may further comprise one or more additional alterations at one or more (e.g. several) other positions in any of the regions described herein.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly. Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant mutant molecules are tested for xanthan lyase activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide.

Disclosed herein is an artificial cutinase enzyme catalyst. Native, wildtype, cutinase has two disulfide bridges. The catalytic power of the artificial cutinase disclosed herein is increased through the deletion of a disulfide bridge within the native, wildtype, cutinase structure. In particular, the disulfide bridge formed between cysteine-31 and cysteine-109 (i.e., C31-C109) is removed while leaving the second disulfide bridge unchanged. Thus, the folding and functional capabilities are maintained while creating an increase in catalytic action.

Wildtype cutinase degrades a naturally occurring plant polyester that has a smaller polymer chain than PET. Cutinase, thus, has a relatively small active site evolved for the smaller plant polyester. Since PET has relatively large polymer chains, cutinase has a difficult time fitting PET into its small active site. The result is an inefficient and slow biodegradation of the plastic. To combat this, embodiments of the disclosure herein increase the amount of folding energy available within the enzyme; which allows for the formation of a larger, more specific, active site that can better fit PET based plastics as a substrate. This was achieved through the targeted deletion of a disulfide bridge, which decreases thermal stability of the enzyme but makes more folding energy available within the enzyme structure. This occurs as the distance/angle constraints imposed on the cysteine molecules by the disulfide bond are broken, thus increasing the availability of movement and folding capabilities of the enzyme. Without subscribing or being bound to a specific mechanism or theory, it is believed that the deletion of the chosen disulfide bridge increases the catalytic power of cutinase due to formation of a larger active site that can made possible by the excess folding energy made available by the deletion.

### Methods

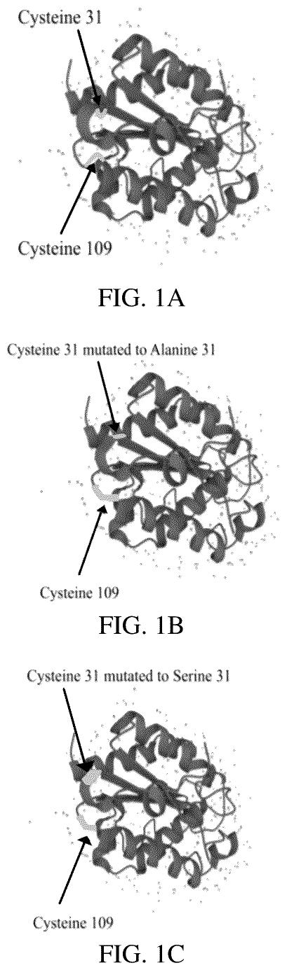

An example DNA sequence of wildtype cutinase is presented as SEQ ID NO: 1 with a corresponding amino acid sequence presented as SEQ ID NO:8. Two mutations of cutinase that deleted the first disulfide bridge at C31-C109 were modeled using the Protein Data Bank Mol* 3D Viewer and its hydrophobic-hydrophilic protein folding model. C31 was altered to the amino acid alanine (A31), which contains an amine group that would not cause incorrect folding or affect protein stability. This mutation was labeled C31A and is represented as a DNA sequence by SEQ ID NO: 6 and an amino acid sequence by SEQ ID NO:9. A model of this change is shown in FIG. 1. The virtual model of the enzyme clearly demonstrated that a change from C31 to A31 did not change the folding sequence of cutinase. This led to the hypothesis that the proposed mutation would be successful in lowering thermal stability and increasing catalytic power. In particular, since cysteine is structurally similar to serine, but does not contain the atom that would attract to Cys 109 (creating the disulfide bridge with Cys 31), it was hypothesized that the mutation to serine would also not cause incorrect folding. This mutation was labeled C31S and is represented as a DNA sequence by SEQ ID NO:7 and an amino acid sequence by SEQ ID NO:10.

There is a second disulfide bridge in cutinase that occurs at C171-C178. Due to the short distance between the composing amino acids, the second disulfide bridge qualifies as a “short-range” disulfide bridge, this contrasts with the “long-range” first disulfide bridge at C31-C109, which spans a much larger distance in the protein fold and contributes more to the structural integrity of the protein. C31-C109 was chosen as the target for deletion due to this long-range/short-range difference. Since the second short range disulfide bridge at C171-C178 covers such a small distance, it does not have a significant influence on the thermal stability of cutinase; thus, it was determined that deleting the short-range disulfide bridge would likely cause no significant structural change to the protein, let alone the active site conformation. In contrast, deletion of the long range C31-C109 disulfide bridge was viewed as more likely to achieve a decrease in thermal stability, thus allowing for a greater amount of folding energy to become available and be reinvested in the active site formation.

Oligonucleotides encompassing Sequences 2-5 (Table 1) were designed using the QuikChange Primer Designer online tool. Primers were desalted upon purchase for purification. They were reconstituted to 100 ng μL⁻¹ in laboratory-grade water and were then aliquoted into three 1-mL samples for use in the site-directed mutagenesis protocol.

TABLE 1
PRIMERS USED FOR SITE-DIRECTED
MUTAGENESIS OF CUNTINASE GENE

| Mutation | Primer (5′-3′) | bp |
|---|---|---|
| C31A | SEQ ID NO: 2: F1 | 32 |
| | ggcaacagcgcgagcgccgcggatgtgatttt | |
| | SEQ ID NO: 3: R1 | |
| | aaaatcacatccgcggcgctcgcgctgttgcc | |
| C31S | SEQ ID NO: 4: F1 | 25 |
| | gcaacagcgcgagctccgcggatgt | |
| | SEQ ID NO: 5: R1 | |
| | acatccgcggagctcgcgctgttgc | |

Oligonucleotides were ordered from Thermofisher Scientific. The QuikChange® Lightning Site-Directed Mutagenesis Kit was purchased from Agilent. BL21 (DE3) competent E. coli cells were purchased from New England Biolabs. A Plasmid Miniprep Kit was purchased from Qiagen. For biodegradation testing, a polyethylene storage bag was used.

A pCWT vector containing the wildtype cutinase gene expressed in BL21 E. coli, was requested from the University Nova de Lisboa, Institute of Chemical and Biological Technology, and was graciously supplied by Dr. Filipa Goncalves. All other reagents were laboratory-grade.

The native cutinase bearing E. coli strain was inoculated into broth and grown overnight. 1.5 mL of the strain was placed in a well plate and grown until absorbance of 0.6 was reached. IPTG was then added to induce the cutinase. After three hours of IPTG activity, a 1×1 cm piece of PET was placed in the well plate containing the cutinase. The samples were incubated at 37° C. for 24 h.

The plasmid was prepared using the Qiagen Plasmid Miniprep Kit. The protocol for the kit was followed. Two samples of plasmid were prepared. The bacterial overnight culture was pelleted by centrifugation at 3,000 g for 3 minutes at room temperature. The pellets were resuspended in 250 μL of Buffer P1 (50 mM Tris Cl, pH 8.0; 10 mM EDTA, 100 μg mL⁻¹ RNAase A; this buffer is used as a resuspension buffer for plasmid DNA purification) and transferred to a microcentrifuge tube. 250 μL of Buffer P2 (200 mM NaOH, 1% SDS w/v; lysis buffer used in plasmid DNA purification) was added and mixed by inverting the tube to lyse the cells. This was centrifuged for 10 minutes at 13,000 rpm in a tabletop microcentrifuge. 800 μL of the supernatant was added to a spin column by pipetting. This was centrifuged for 60 seconds and flow-through was discarded. The spin column was washed by adding 0.75 mL of Buffer PE (composition proprietary to Qiagen), the wash buffer. This was centrifuged for 60 seconds and flow-through was discarded. This spin column was centrifuged again for 1 minute to remove any residual wash buffer. The column was placed in a clean microcentrifuge tube. To elute the DNA, 50 μL of Buffer EB (10 mM Tris-CL, pH 8.5) was added to the center of the spin column. This stood for 1 minute and was then centrifuged for 1 minute.

After the plasmid DNA of cutinase was retrieved from the bacteria, a standard Nanodrop DNA quantification test was completed to determine if there was sufficient DNA in the sample. The Nanodrop test indicated that the first sample contained 3.3 ng μL⁻¹ of DNA. The second sample contained about 7.2 ng μL⁻¹.

Two mutagenesis reactions were prepared. Plasmid DNA yielded from the Qiagen Miniprep was used in the reactions. The protocol from the Agilent QuikChange® Lightning Site-Directed Mutagenesis Kit was followed for these reactions (Tables 2, 3).

TABLE 2
PREPARED SITE-DIRECTED MUTAGENESIS REACTION #1

| Tube # | Buffer | Primer F/R* | dNTP mix | Quik solution | Plasmid | dH2O** | Enzyme |
|---|---|---|---|---|---|---|---|
| 1 (Control) | 5 μL | 1.25 μL | 1 μL | 1.5 μL | 5 μL | 34 μL | 1 μL |
| 2 (C31A) | 5 μL | 1.25 μL | 1 μL | 1.5 μL | 15 μL | 24 μL | 1 μL |
| 3 (C31A) | 5 μL | 1.25 μL | 1 μL | 1.5 μL | 7 μL | 32 μL | 1 μL |
| 4 (C31S) | 5 μL | 1.25 μL | 1 μL | 1.5 μL | 15 μL | 24 μL | 1 μL |
| 5 (C31S) | 5 μL | 1.25 μL | 1 μL | 1.5 μL | 7 μL | 32 μL | 1 μL |

*F/R stands for forward/reverse. In this experiment, primers labeled “antisense” are assumed to be the reverse primers.
**Each reaction measured 50 μL in volume. This is the reason for the differing amounts of plasmid and water in each reaction.

TABLE 3
PREPARED SITE-DIRECTED MUTAGENESIS REACTION #2

| Tube # | Buffer | Primer F/R | dNTP mix | Quik solution | Plasmid | dH2O | Enzyme |
|---|---|---|---|---|---|---|---|
| No control* | — | — | — | — | — | — | — |
| 1 (C31A) | 5 μL | 1.25 μL | 1 μL | 1.5 μL | 15 μL | 24 μL | 1 μL |
| 2 (C31S) | 5 μL | 1.25 μL | 1 μL | 1.5 μL | 7 μL | 32 μL | 1 μL |

*No control sample was prepared because the first mutagenesis yielded transformants of the control, but no transformants of the mutations.

These reactions were cycled following the parameters in the kit protocol (Table 4).

TABLE 4
THERMOCYCLING PROTOCOL FOR
MUTAGENESIS REACTIONS

| Segment | Cycles | Temperature | Time | |
|---|---|---|---|---|
| 1 | 1 | 95° C. | 2 | min |
| 2 | 18 | 95° C. | 20 | s |
| | | 60° C. | 10 | s |
| | | 68° C. | 30 s per kb of | |
| | | | plasmid length* | |
| 3 | 1 | 68° C. | 5 | min |

*Plasmid length is about 6 kb, cycled for about 3 minutes*

Transformation into BL21 (DE3) E. coli

Following the preparation of the first mutagenesis reaction, a first transformation (Table 5) was attempted following the protocol outlined in the QuikChange® Site-Directed Mutagenesis Kit Protocol. The provided control was also tested in this transformation. While the control samples did transform, no colonies were yielded for the mutations.

A second transformation (Table 5) attempt increased the amount of plasmid DNA in the reaction from 2 μL to 10 μL to increase the possibility of a successful yield. This transformation did not yield any colonies.

A third transformation (Table 5) attempt increased the amount of plasmid DNA in the reaction from 10 μL to 20 μL. This transformation did not yield any colonies.

TABLE 5
CONTENTS OF TRANSFORMATIONS 1-4

| Transformation | Competent | β-mercapto- | Dpn-treated | NZY + |
|---|---|---|---|---|
| # | cells | ethanol | plasmid DNA | Broth |
| 1 | 45 μL | 2 μL | 2 μL | 500 μL |
| 2 | 45 μL | 2 μL | 10 μL | 500 μL |
| 3 | 45 μL | 2 μL | 20 μL | 500 μL |
| 4 | 90 μL | 2 μL | 10 μL | 500 μL |

Between the third and fourth transformation, further research was done on troubleshooting for site-directed mutagenesis transformations. Indications were found that maintaining a 1:10 ratio of plasmid DNA to competent cells in a transformation reaction may maximize transformation efficiency. Therefore, in the fourth transformation (Table 5), plasmid DNA was decreased from 20 μL to 10 μL, and the competent cells were increased from 45 μL to 90 μL. The fourth transformation did not yield any colonies.

Since no colonies were yielded after the fourth transformation, the second mutagenesis reaction was prepared. It was hypothesized that the problem lay in the first mutagenesis reaction, not the transformation reactions. Due to the amount of remaining plasmid, only one plasmid containing each mutation was transformed instead of two. No control was prepared for the second mutagenesis reaction.

The fifth transformation (Table 6) was prepared using the second mutagenesis reaction. Each mutation was plated twice, once with 45 μL of competent cells and once with 90 μL of competent cells. The fifth transformation yielded colonies in three of the four plates.

TABLE 6
CONTENTS OF TRANSFORMATION 5

| | Competent | β-mercapto- | Dpn-treated | NZY + |
|---|---|---|---|---|
| Plate # | cells | ethanol | plasmid DNA | Broth |
| 1 (C31A) | 45 μL | 2 μL | 2 μL | 500 μL |
| 2 (C31A) | 90 μL | 2 μL | 10 μL | 500 μL |
| 3 (C31S) | 45 μL | 2 μL | 2 μL | 500 μL |
| 4 (C31S) | 90 μL | 2 μL | 10 μL | 500 μL |

After reactions were prepared according to the kit protocol, they were incubated at 37° C. for one hour with shaking at 225 rpm. After incubation, the reactions were plated on TSA (tryptic soy agar) plates with ampicillin; 250 μL of each reaction was plated. The plates were incubated overnight at 37° C.

Two colonies of each mutation, C31A and C31S (exemplified by SEQ. ID. NO: 09 and SEQ. ID. No: 10), were inoculated into broth and grown overnight. 1.5 mL of each mutation was placed in a well plate and grown until absorbance of 0.6 was reached. IPTG (isopropyl β-D-1-thiogalactopyranoside) was then added to induce the cutinase. After three hours of IPTG activity, a 1×1 cm piece of PET was placed in the well plate containing the cutinase. The samples were incubated at 37° C. for 24 hours.

### Results

The mass of each plastic piece prior to and following incubation with the wildtype cutinase strain was recorded (Table 7).

TABLE 7
MASS REDUCTION OF PET FOR CUTINASE
ASSAY (WILDTYPE STRAIN)

| Strain | Original Mass | Final Mass |
|---|---|---|
| Wild-type cute + 1 | .004 g | .003 g |
| Wild-type cute + 2 | .005 g | .003 g |

The mass of each plastic piece prior to and following incubation with the mutated cutinase strains were recorded (Table 8). A media-only control was also completed (Table 8).

TABLE 8
MASS REDUCTION OF PET FOR CUTINASE ASSAY
(MEDIA CONTROL AND MUTATED STRAINS)

| Strain | Original Mass | Mass after Incubation |
|---|---|---|
| Media control | .004 g | .004 g |
| C31A-1 | .004 g | .001 g |
| C31A-2 | .004 g | .001 g |
| C31S-1 | .005 g | 0.00 g |
| C31S-2 | .005 g | 0.00 g |

The percent change for the tested plastic samples was calculated between the original mass of the plastic and the mass following incubation (Table 9).

TABLE 9
PERCENT CHANGE OF PLASTIC MASS

| Strain | Percent change of PET sample |
|---|---|
| Media control | 0% decrease |
| Wild-type cute + 1 | 25% decrease |
| Wild-type cute + 2 | 40% decrease |
| C31A-1 | 75% decrease |
| C31A-2 | 75% decrease |
| C31S-1 | 100% decrease |
| C31S-2 | 100% decrease |

In an additional test, enzyme samples were incubated with a massed 3×3 cm of PET in a shaker at 37° Celsius for 48 h and then massed again (Table 10).

TABLE 10

| Sample | IPTG? | Mass Prior | Mass After |
|---|---|---|---|
| Media control | − | .031 g | .031 g |
| Wild-type cute+ | − | .028 g | .028 g |
| C31A | − | .028 g | .027 g |
| C31S | − | .032 g | .031 g |
| Wild-type cute+ | ++ | .033 g | .032 g |
| C31A | ++ | .026 g | .019 g |
| C31S | ++ | .033 g | .000 g |

C31A percent change: 37%
C31S percent change: 100%

In another additional test, enzyme samples were again incubated with a massed 3×3 cm of PET in a shaker at 37° C. for 48 h and then massed again. PET was sampled from a plastic water bottle and a zippered storage bag (to test the enzyme on different PET samples of different durability). Results are presented in Table 11.

TABLE 11

| Sample | IPTG? | Mass Prior | Mass After |
|---|---|---|---|
| Wild-type cute + bot | − | .018 g | .016 g |
| C31A bot | − | .015 g | .015 g |
| C31S bot | − | .012 g | .012 g |
| Wild-type cute + bot | ++ | .016 g | .015 g |
| C31A bot | ++ | .020 g | .015 g |
| C31S bot | ++ | .015 g | .000 g |
| Wild-type cute + zip | − | .013 g | .012 g |
| C31A zip | − | .012 g | .012 g |
| C31S zip | − | .017 g | .015 g |
| Wild-type cute + zip | ++ | .015 g | .015 g |
| C31A zip | ++ | .013 g | .012 g |
| C31S zip | ++ | .014 g | .010 g |

C31A bottle percent change: 25%
C31S bottle percent change: 100%
C31A zippered bag percent change: 8%
C31S zippered bag percent change: 29%

In a final additional test, enzyme samples were again incubated with a massed 3×3 cm of PET in a shaker at 37° C. for 48 h and then massed again (Table 12).

TABLE 12

| | Mutation | | | | |
|---|---|---|---|---|---|
| Tube # | exposed | IPTG+/− | Mass prior | Mass after | % Change |
| 1 | Wild-type | − | .013 g | .013 | 0% |
| 2 | Wild-type | ++ | .011 g | .016 | 45%+ |
| 3 | Wild-type | ++ | .024 g | .023 | 4%− |
| 4 | Wild-type | ++ | .023 g | .025 | 9%+ |
| 5 | C31A | − | .021 g | .021 | 0% |
| 6 | C31A | ++ | .018 g | .017 | 6%− |
| 7 | C31A | ++ | .011 g | .010 | 9%− |
| 8 | C31A | ++ | .015 g | .014 | 7%− |
| 9 | C31S | − | .011 g | .012 | 9%+ |
| 10 | C31S | ++ | .014 g | .008 | 43%− |
| 11 | C31S | ++ | .012 g | .009 | 25%− |
| 12 | C31S | ++ | .017 g | .006 | 65%− |

The above presented results support that the deletion of the C31-C109 disulfide bridge increases enzymatic activity for PET degradation. In all instances tested, a mutant strain outperformed the wildtype with the C31S mutation typically outperforming the C31A mutation. The deleted disulfide bridge also results in a mutant cutinase that is more active at standard room temperature, thus lowering the energy requirements for industrialization.

Thus, embodiments of the cutinase contained herein are suitable for both industrial and residential degradation of PET. In particular, using the primers and techniques herein described, embodiments of the invention may be used to mass produce the cutinase enzyme in purified or substantially purified form, using standard techniques of primer preparation, plasmid amplification, transfection, bioreactor growth and protein purification. In embodiments of the invention, a broad range of hosts or cell types may be cultivated in one or more bioreactors or fermenters to produce the artificial cutinase. Conditions in such bioreactors or fermenters may be tailored to the organisms, or combinations thereof, within them. Such tailoring is within the scope of normal practice to those who operate and scale up bioreactors for production purposes. Cells such as mammalian cell lines, insect cells, plant cells, or stem cells may be used for production. Or, in other embodiments, microorganisms such as bacteria, yeasts, fungi, and algae may be mutated using common techniques known in the art to produce the cutinase. In certain embodiments the cells may be BL21 E. coli. One or more embodiments of the disclosure can include vectors, such as cDNA plasmids, encoding embodiments of the artificial cutinase enzyme and used to transfect hosts for production of the artificial cutinase enzyme.

A typical bioreactor run may include the steps of preculture, bioreactor preparation, inoculation, cultivation, culture harvest, downstream processing, and subsequent bioreactor cleaning for reuse. Depending on organism, host, or cell chosen, those of skill in the art can readily supply parameters for production, including: aerobic or anaerobic conditions; temperature; pH; dissolved oxygen or other gasses; agitation mode and intensity; and other parameters such as are known in the art. One or more bioreactors may be employed in a manufacturing plant to bulk produce the invention. Embodiments of the invention may be grown internally to an organism, requiring lysis, or may be secreted/excreted for downstream processing.

Downstream processing includes one or more steps to harvest and purify the artificial cutinase to one or more degrees of purity for usage. Cells may be separated from the medium, and possibly disrupted to release product. Various chromatographic methods, such as: affinity, ion exchange, size exclusion, reverse phase, hydroxyapatite, and hydrophobic interaction chromatography may be employed. Centrifugation, such as: ultracentrifugation, differential centrifugation, gradient centrifugation, isopycnic centrifugation (equilibrium centrifugation), may also be employed. Precipitation techniques such as ammonium sulfate precipitation and/or salting out. Further separation techniques include liquid-liquid extraction, protein tagging (e.g., His-tag and GST-tagging), dialysis, sonication, gravity settling, and various forms of filtration (e.g., single pass, tangential flow) all may be used to separate and purify embodiments of the invention.

In practice, one or more embodiments of the invention may be in the form of a solution containing the artificial cutinase enzyme and one or more stabilizers. Alternatively, the protein may be lyophilized either alone, substantially purified, purified, or with additional stabilizers or other solution components. A solution containing embodiments of the invention may be stored at room temperature, 15-30° C., 4° C., 0° C., or at −20 to −80° C. depending on composition and factors such as protein purity, concentration, pH, and other factors as known to those of skill in the art. One or more cryoprotectants such as glycerol or ethylene glycol may be used to help stabilize the solution. Additional stabilizers may include protease inhibitors, phosphatase inhibitors, antimicrobial agents (e.g., sodium azide, thimerosal, benzalkonium), metal chelators (e.g., EDTA), and reducing agents (e.g., dithiothreitol, 2-mercaptoethanol). Various pH buffers, such as Tris buffer, may also be used as required. Still other embodiments may place lyophilized pure or substantially purified artificial cutinase enzyme as a composition into one or more containers for storage and subsequent dispersal into one or more solutions in accordance with the below described methods.

In still other methods of practice, a solution containing an embodiment of the artificial enzyme above is exposed to one or more PET containing articles. The solution may be sprayed onto the PET containing articles, or in an alternative, the articles may be immersed in the solution. The articles may be agitated to enhance degradation. One or more sprays or immersions may be necessary to fully obtain decomposition. In some embodiments the PET containing articles may be shredded to increase particle surface area for exposure. In still other embodiments the solution may contain additional surfactants (e.g., a polysorbate, a detergent, etc.).

Upon completion of degradation the resulting slurry may be further fed into one or more treatment chambers for chemical treatment (e.g., pH balancing, metal removal), settling, or bacterial digestion. In the alternative, the slurry may be discharged to the environment.

In still another method of practice an open-air bioreactor populated with an organism transfected to produce the artificial cutinase disclosed herein is provided. Upon reaching a critical population density the bioreactor may be exposed to one or more PET containing articles or pieces. The organism may be lysed to release the cutinase if necessary using means such as sonication, mechanical agitation, lysis enzymes, etc. such as are known in the art. In still another embodiment, the lysis may occur before PET article exposure. In this way, concerns regarding cutinase storage or purification are mitigated and only a small seed population of transfected organism need be kept to repopulate and reuse the bioreactor. Although many of the cell and organisms above described, algae, yeast, and fungal cells may be particularly suitable for such embodiments of the invention.

Finally, the written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

Where a combination is disclosed, it is understood that each possible sub-combination of the elements of that combination is also disclosed. Conversely, where different elements or groups of elements are individually disclosed, combinations thereof are also disclosed.

Where elements are presented in list format or as alternative members of a group (e.g., a Markush group), it is understood that each possible subgroup of the elements is also disclosed, and any one or more elements can be removed from that list or group.

Where a range of values is recited, it is understood that each intervening integer value and each fraction thereof, as well as each subrange, between the recited upper and lower limits of that range are specifically disclosed. Where a value has an inherent limit, that inherent limit is specifically disclosed. Where a value is explicitly recited, it is understood that values which are about the same as the recited value are specifically disclosed.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. An artificial cutinase polypeptide wherein the first disulfide bridge is deleted.

2. The artificial cutinase polypeptide of claim 1 wherein the cutinase polypeptide is lyophilized.

3. The artificial cutinase polypeptide of claim 1 wherein the cutinase polypeptide is in a solution with at least one stabilizer.

4. An artificial vector encoding the artificial cutinase polypeptide of claim 1.

5. The artificial cutinase polypeptide of claim 1 wherein C31 is mutated to an Alanine.

6. The artificial cutinase polypeptide of claim 1 wherein C31 is mutated to a Serine.

7. An artificially cultivated organism transfected with the artificial vector of claim 4.

8. The artificially cultivated organism of claim 7 wherein the organism is grown in a bioreactor.

9. A method of PET article degradation, comprising exposing a PET article to the artificial cutinase polypeptide of claim 1.

10. The method of claim 9 wherein the artificial cutinase polypeptide is contained and/or secreted by the artificially cultivated organism of claim 7.

11. The method of claim 9, wherein the exposure of the PET article occurs within an open-air bioreactor.

12. The method of claim 9, wherein the exposure is accomplished via a solution containing the artificial cutinase polypeptide.

13. The method of claim 12, wherein the solution further comprises at least one selected from the group of: a surfactant, a pH buffer, and an enzyme stabilizer.

14. The method of claim 9 wherein, after degradation, the resulting waste stream is subjected to further treatment.

15. A nucleic acid sequence comprising SEQ ID NO: 1 or at least 40% sequence identity with a conserved active site, wherein C31 is mutated to an Alanine or Serine.

16. An artificial cutinase polypeptide having the polypeptide sequence of SEQ ID NO: 9.

17. An artificial cutinase polypeptide having the polypeptide sequence of SEQ ID NO: 10.


SEQUENCE LISTING (5 sequences)

SEQ ID NO: 1 - protein, 934 aa, Escherichia coli
  DISULFID 31: note = First Disulfide Bridge
  DISULFID 171: note = Second Disulfide Bridge
NNNNNNNNNNNNNNNNTNNGCTAGTTNNTGCTCNGCGGTGGCAGCAGCCAACTCAGCTTCCTTTCGGGCTTTGTTAGCAGCCGGATCTCAGTGGTGGTGGTGGTGGTGCTCGACATCCTCGGGGTCTTCCGGGGCGAGTTCTGGCTGGCTAGCCCGTTTGATCTCGAGTGCGGCCGCAAGCTTGTCGACGGAGCTCGCAGCAGAACCACGGACAGCCCGAACCTTCTCGATGAGGAACTCAGGGGCAGGGCCACGAGCATCAGGACCATAAGCCAAGTGAGGTGCAGCAACGATCAAGCTACCAGTACAAACGAGATCCCCTGTATTGCAGAAGACCTTGGTCCTGTCGGCAGGGTAGTTGGGGATTCGGCCACGGTTCTGTAGGTTCTTGGTGTAGCCGAACAGAACAGTTCCGGCGATCTTGTCACGAATGGCCGAGTCGAGGTCCTCGATGGAGGCGGCTGCAAGTGCAGCACCCTGGCTGTAGCCACCGGCGATCAAAGTCGCGTCAGGGCACTTGGTGTTGGCCTGCTGGAAGAGACCGAGCATCTCCCTGATTGCGGCGCTAGAGGTTCCGCGAGGGAGAGCATTGTCTCCAAGAGTGGCTCGGTAGGCACCGCCAACGCCCTGAATCCAGACACCGTCCTTGCCGAAGGCGGACTCAAGGTTGGAGGCAATGCTAGGACCGAGAGTTCCCAAGTTGCCCGTCTCTGTTGAACCTCGGGCATAAATGAAGATGACATCGGCGCANNAAGCGCTANTGNCNTTGATCAGATCGTCGCGAGTTGTTCTACCAAGCTGGCGCGCCTCAAGCTCCTGGGCAGGTTAGAAGTNNCGCGGCTTTTGTCACNGGGTAAACAGTAACGGTAAGAGTGNCNAGTGCATAGTGCTTTGTTTCATGGGATCCGAATTATTCCNANATCCNNGGCCATCN

SEQ ID NO: 2 - DNA, 32 bp, synthetic construct
  source 1..32: mol_type = other DNA
ggcaacagcgcgagcgccgcggatgtgatttt

SEQ ID NO: 3 - DNA, 32 bp, synthetic construct
  source 1..32: mol_type = other DNA
aaaatcacatccgcggcgctcgcgctgttgcc

SEQ ID NO: 4 - DNA, 25 bp, synthetic construct
  source 1..25: mol_type = other DNA
gcaacagcgcgagctccgcggatgt

SEQ ID NO: 5 - DNA, 25 bp, synthetic construct
  source 1..25: mol_type = other DNA
acatccgcggagctcgcgctgttgc